(19) United States
(12) Patent Application Publication
(10) Pub. No.: US 2018/0333249 A1
(43) Pub. Date: Nov. 22, 2018

(54) DEGRADABLE INTESTINAL ANCHOR

(71) Applicant: THE REGENTS OF THE UNIVERSITY OF CALIFORNIA, Oakland, CA (US)

(72) Inventors: James Dunn, Santa Monica, CA (US); Benjamin Wu, San Marino, CA (US)

(73) Assignee: THE REGENTS OF THE UNIVERSITY OF CALIFORNIA, Oakland, CA (US)

(21) Appl. No.: 15/973,062

(22) Filed: May 7, 2018

Related U.S. Application Data
(63) Continuation of application No. PCT/US2016/061598, filed on Nov. 11, 2016.
(60) Provisional application No. 62/254,160, filed on Nov. 11, 2015.

(51) Int. Cl. A61F 2/04; A61F 2/93; A61B 90/00

(52) U.S. Cl. CPC A61F 2/04; A61F 2/93; A61B 90/02; A61F 2002/828; A61F 2210/0004; A61F 2220/005; A61F 2002/045

(57) ABSTRACT

An organ lengthening device comprising a spring-like structure, wherein the surface of the device is covered with micron-size anchors such as hooks, studs or wires made from a biodegradable polymer. The anchors are configured to engage the surface of the organ so that the device will be anchored to the organ. The device, which is inserted into the organ in a compressed position, gradually lengthens over time, thereby lengthening the organ, wherein the anchors are configured to degrade away and eventually allow the device to become disengaged from the organ.

### CROSS-REFERENCE TO RELATED APPLICATIONS

This application is a 35 U.S.C. § 111(a) continuation of PCT international application number PCT/US2016/061598 filed on Nov. 11, 2016, incorporated herein by reference in its entirety, which claims priority to, and the benefit of, U.S. provisional patent application Ser. No. 62/254,160 filed on Nov. 11, 2015, incorporated herein by reference in its entirety. Priority is claimed to each of the foregoing applications.

The above-referenced PCT international application was published as PCT International Publication No. WO 2017/083696 on May 18, 2017, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to an organ lengthening device, and more particularly to an organ lengthening device that is configured for distraction enterogenesis of intestinal segments.

### 2. Background Discussion

Short bowel syndrome (SBS), also known as short gut syndrome, occurs in patients with insufficient length of intestine to maintain normal digestion and absorption. Current surgical therapies include lengthening, transit-slowing procedures, or bowel transplantation, but patient selection is limited and long-term benefits are not clearly shown.

Tissue expander devices have been used to apply mechanical force to lengthen intestinal tissue as a way to treat this condition. However, current methods that anchor devices to the gastrointestinal tract are not degradable and only secure the ends of the device to the organ. An example can be found in U.S. Pat. No. 9,138,336 issued on Sep. 22, 2015.

### BRIEF SUMMARY

This disclosure pertains to methods and devices for lengthening a hollow internal organ that do not rely on anchoring the ends of the device to the organ during the expansion process. Alone, or in combination with means for anchoring the ends, methods and devices according to embodiments of the technology described herein employ surface modifications for anchoring the tissue between the ends of the device.

By way of example, and not of limitation, a lengthening device according to embodiments of the technology described herein employ specialized modification of the surface that allow the device to be adherent to the organ for a desired period of time. This specialized surface is degraded during that time period so that the device becomes detached from the organ after the lengthening is accomplished.

In one embodiment of the specialized modification, the surface of the device is covered with micron-size features, or micro-anchors, such as hooks or wires made from a biodegradable polymer. These micro-anchors engage the surface of the organ so that the device attaches to the organ. The device, which is inserted into the organ in a compressed position, gradually lengthens over time and, during that time, the micro-anchors degrade away and eventually allow the device to become disengaged from the organ.

In another embodiment of the specialized modification, the surface of the device is sprayed or coated with an adhesive mixed with a biodegradable polymer. The device is then “glued” to the surface of the organ until the lengthening has completed. The adhesive coating degrades away over time to allow detachment of the device from the organ afterwards.

In another embodiment, the device comprises specialized ends to support the engagement of the device to the organ.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 shows a perspective view of an organ lengthening device in an uncompressed configuration in accordance with the present description.

FIG. 2 shows a configuration of an organ lengthening device in an uncompressed configuration having barb-shaped (i.e. tapered) studs that are angled to point in opposing directions.

FIG. 3 shows a perspective view of an organ lengthening device in an uncompressed configuration having cylindrical studs that are angled to point in a single axial direction.

FIG. 4 shows a perspective view of an organ lengthening device in an uncompressed configuration having opposing crown-shaped anchors disposed on opposite ends of the device.

FIG. 5 shows a perspective view of a ratchet-type end anchor according to an embodiment of the technology described herein.

FIG. 6 shows a schematic diagram of a modified spring surface with surface anchors according to an embodiment of the technology described herein.

FIG. 7 is a profile-view micrograph of the fins and spring of an embodiment of the technology described herein.

FIG. 8 shows a schematic view of a device compressed into an absorbable retaining element for delivery according to an embodiment of the technology described herein.

FIG. 9 is an image illustrating intact histology of an intestine lengthened by a device according to an embodiment of the technology described herein.

FIG. 10A shows a graph of increased crypt depth of in-continuity jejunum in comparison to normal jejunum.

FIG. 10B shows a graph of muscularis thickness of in-continuity jejunum in comparison to normal jejunum.

FIG. 11 shows a light microscopy image of hematoxylin and eosin-stained normal (non-lengthened) jejunum at 4× magnification.

FIG. 12 shows a light microscopy image of hematoxylin and eosin-stained lengthened jejunum at 4× magnification.

### DETAILED DESCRIPTION

The present description is directed to an organ lengthening device employing a specialized modification of the surface of a compressible/extendable spring structure that allows the device to be adhered to the organ for a desired period of time. In a preferred embodiment, the surface is configured to degrade after a specified time period, so that the device will become detached from the organ after the desired lengthening is accomplished. The length of time for the degradation to take place can range, for example, from about three weeks to about three months, and preferably up to about six weeks. Degradation characteristics are generally a function of the biodegradable materials used, and their degradation characteristics over the desired period of time.

In a preferred embodiment of the systems and methods of the present description, an organ lengthening device is configured for distraction enterogenesis of intestinal segments as a novel treatment for patients with short bowel syndrome (SBS).

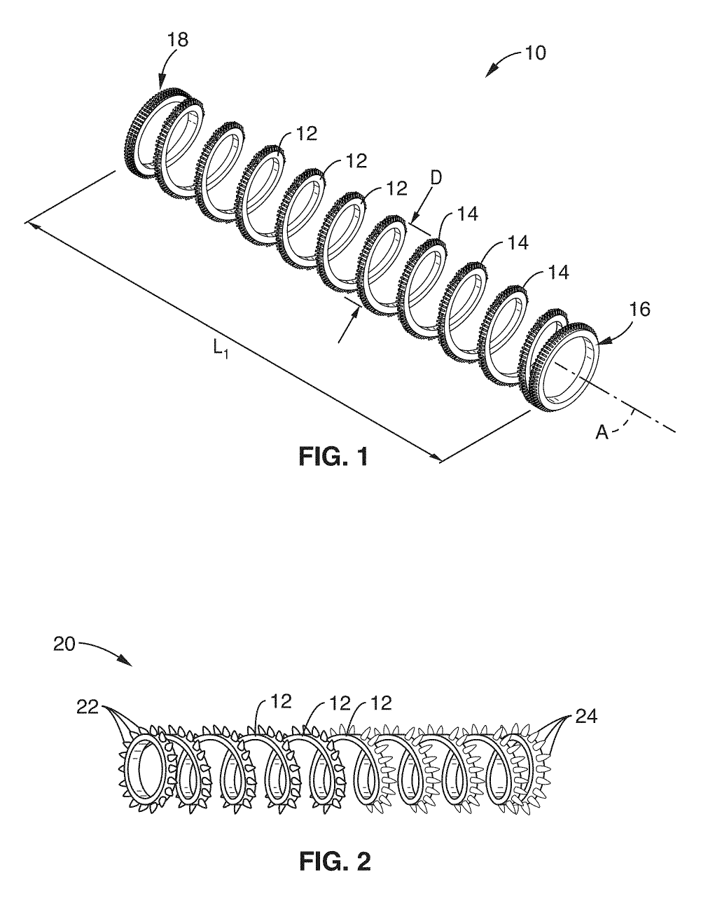

FIG. 1 shows a perspective view of an organ lengthening device 10 in an uncompressed configuration in accordance with the present description. Device 10 comprises a spring-shaped structure composed of a plurality of coils 12 forming a diameter D selected to match or be slightly larger than the internal wall of the organ to be lengthened, and having a central axial channel along axis A allowing normal function of the organ when placed at the treatment location (e.g. a luminal organ such as the intestines, esophagus or vagina (not shown)). The device 10 has an uncompressed or free-state length L₁ between first end 16 and second end 18 that corresponds with the desired lengthening of the organ. Each of the coils 12 comprise a plurality of micro-anchors 14 configured to engage the internal wall of the organ to provide purchase at the target luminal segment for the applied forces on the organ. When compressed, the coils provide an outward force on the organ at the target luminal segment along the axis A of the coils 12. In the embodiment shown in FIG. 1, the micro-anchors 14 comprise a plurality of studs disposed on the outer surface of the coils.

The micro-anchors 14 may be configured via a number of varying parameters that are selected for their unique characteristics in engaging the anatomy of the lumen wall. For example, micro anchors 14 may vary in sizing (e.g. length of the surface anchors (e.g., 50 to 500 microns), diameter of the surface anchors (e.g., 50 to 200 microns)), density, location, shape (e.g. straight, curled, and hooked, tapered, blunt, etc.) angulation (e.g., 10 to 90 degrees in either direction with respect to the axis A), material (e.g., polycaprolactone, polyglycolic acid, or other like biodegradable polymers or materials), etc.

FIG. 2 shows an exemplary configuration of an organ lengthening device 20 in an uncompressed configuration having larger, barb-shaped (i.e. tapered) studs 22, 24 that are angled to point in opposing directions. This configuration angles the studs 22, 24 into the luminal tissue to oppose the axial loading/forces exerted by the spring coils 12 on the tissue.

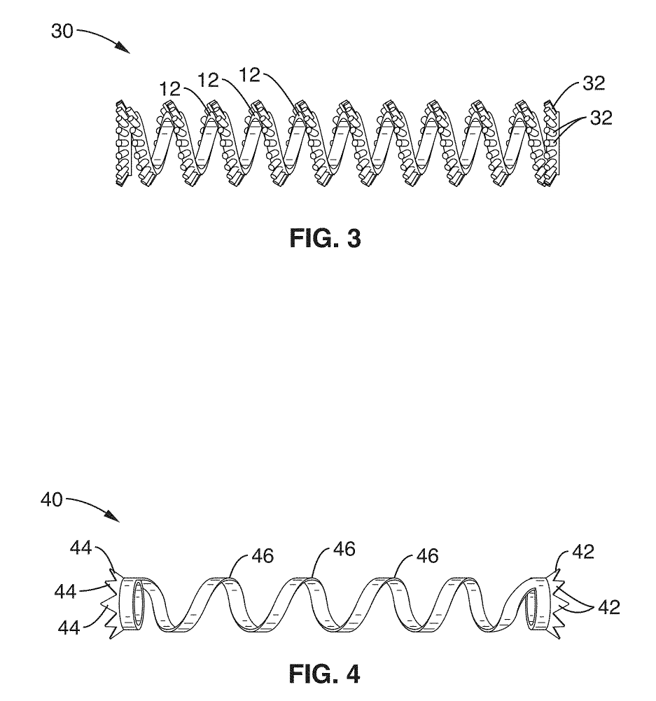

FIG. 3 shows a perspective view of an organ lengthening device 30 in an uncompressed configuration having larger, cylindrical studs 32 that are angled to point in a single axial direction. This configuration angles the studs 22, 24 into the luminal tissue to oppose the axial loading/forces exerted by the spring coils 12 on the tissue.

Micro-anchors 14 may also be selectively positioned at specific regions or locations on the device or coils, and particularly at ends 16, 18 of the coils 12. FIG. 4 shows a perspective view of an organ lengthening device 40 in an uncompressed configuration having opposing crown-shaped anchors 42/44 disposed on opposite ends of the device. The crown-shaped anchors 42/44 are angled to point in opposing directions into the luminal tissue to oppose the axial loading/forces exerted by the spring coils 46 on the tissue. The inner coils 46 are free from anchors, allowing tissue to translate, or distend, freely and unimpeded with respect to the inner coils 46.

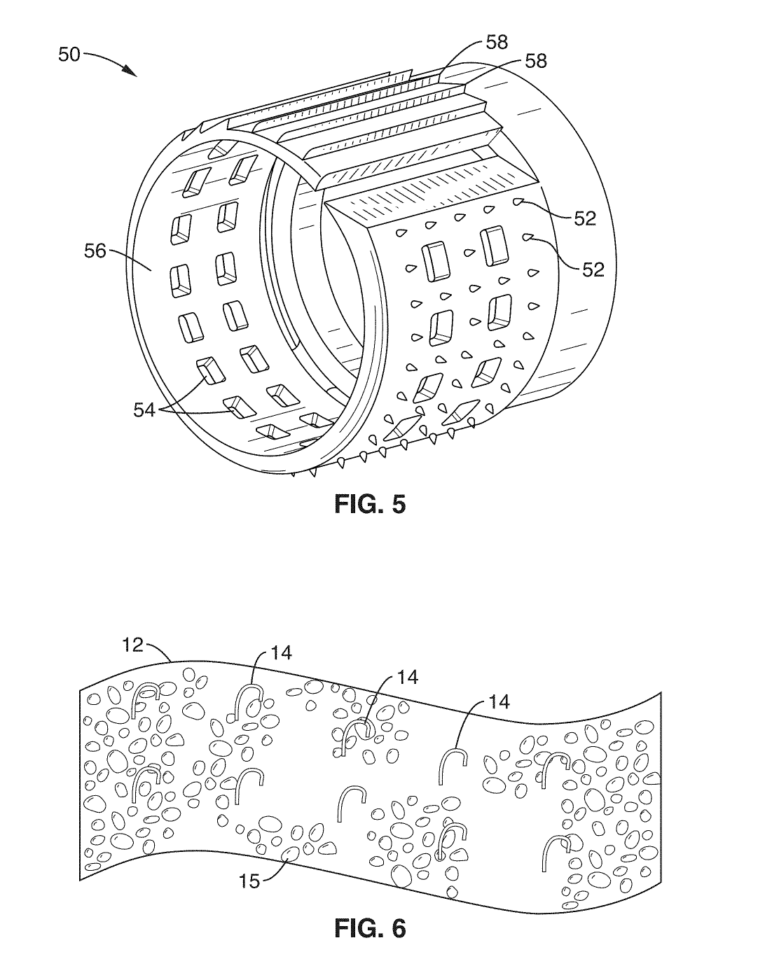

FIG. 5 shows an end cap configuration 50 to be used on opposing ends of a spring 15. End cap 50 comprises a ratchet-type anchor configured to engage the lumen wall with a free end 56 that ratchets open along the lumen wall via teeth 58. Openings 54 and allow the lining cells of the intestine to grow into it through the end cap 50. The barbs 52 on the surface are adapted to fit into the openings 54 on the upper portion of the figure for ratcheting the anchor to the desired outer diameter.

FIG. 6 shows a schematic diagram of a modified spring surface with surface anchors according to an embodiment of the technology described herein. As shown in the detailed view, the outer, or abluminal, surface 15 of the spring 12 is covered with micron-size anchors 14, which may include one or more of studs, hooks, wires or fins (surface anchors) that are preferably made from a biodegradable polymer. These micro-anchors 14 are configured to engage the inner tissue surface of the organ so that the device will be anchored to the organ after insertion. The device 10, 20, 30 40 (see FIG. 1 through FIG. 4), which in an operable configuration is inserted into the organ in a compressed position, is configured to engage the inner walls of the organ gradually lengthen over time to its free state. As the outer abluminal surface 15, and in particular micro anchors 14 are engaged with the inner wall of the organ, the inner wall section of the organ adjacent the springs 12 is distended as the springs expand. During that time, the surface anchors 14 are configured to gradually degrade away and eventually allow the device 10 to become disengaged from the organ.

In another embodiment, the surface 15 of the springs 12 may be coated with an adhesive mixed with a biodegradable polymer. As a result, the device will be “glued” to the inner surface of the organ until the lengthening has completed. The adhesive coating is configured to degrade away over time to allow detachment of the device from the organ afterwards. Various biodegradable adhesive materials known in the art may be employed, including, but not limited to, mussel foot protein, peptides and even superglue.

It is also appreciated that the coils 12 may be comprised of a 2-ply structure (not shown), wherein a first outer abluminal layer comprises an anchoring configuration (e.g. with micro anchors 14, adhesive, or both) that is biodegradable, and a second inner adluminal layer comprises a different material (e.g. shape memory metal such as nitinol or the like) that may be more stiff, non-biodegradable, and configured to pass through the system once lengthening is achieved.

### EXAMPLE 1

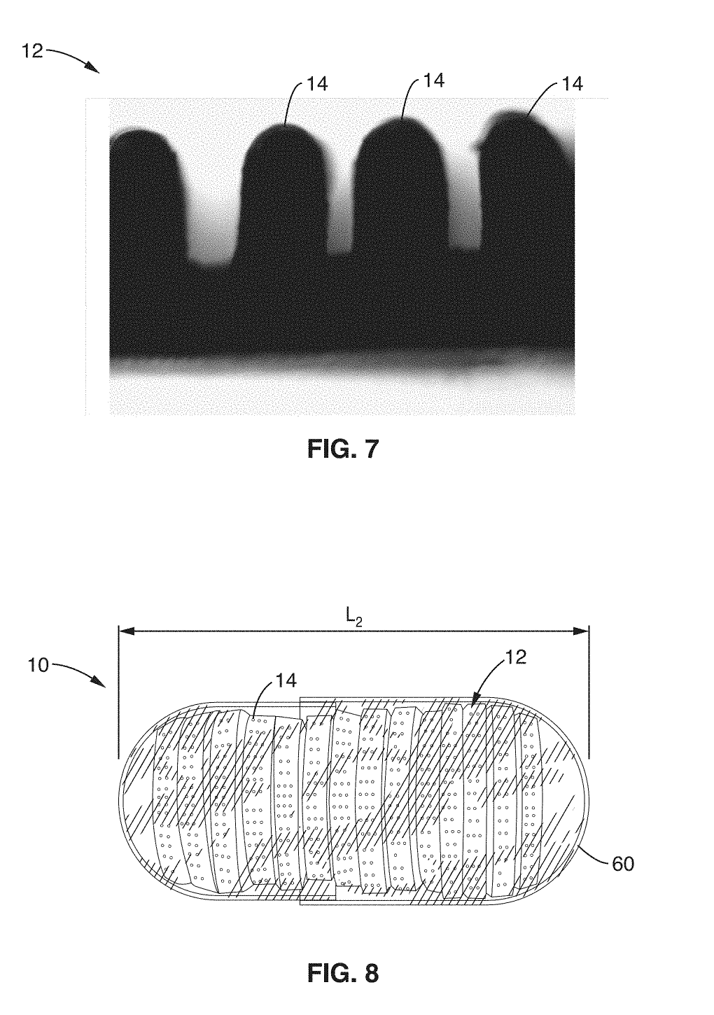

A device based on the above-detailed embodiments was fabricated and tested in pigs. The device was made by fixing multiple microscopic anchors or fins to the surface of the spring. These features were produced by molding polymers. A profile-view micrograph of the fins 14 and spring 12 is shown in FIG. 7. FIG. 8 shows a schematic view of a device 10 with anchors 14 evenly distributed over the length of the coils 12 compressed into an absorbable retaining element 60 for delivery of the compressed device. The device has a compressed length L₂ that is delivered into a target region of the organ, wherein the retaining element dissolves to release the device 10 and engage springs 12 with the lumen walls. The device 10 then expands toward the non-compressed length L₁, distending the adjacent lumen with it.

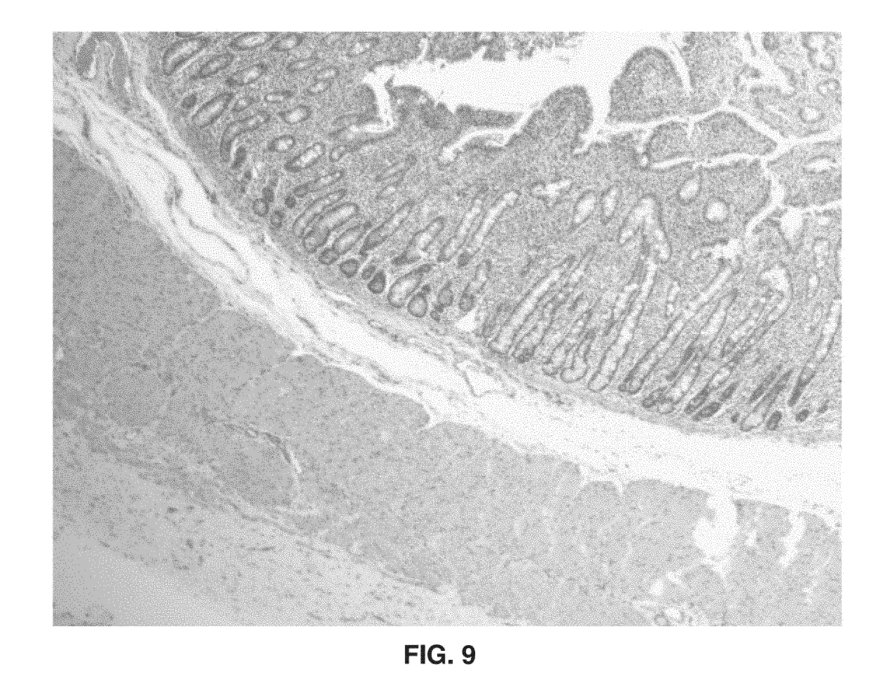

The device was inserted into the lumen of a porcine intestine and observed after two weeks of use. After time, the device degraded and passed through the intestine without causing blockage. As illustrated in the image of the intestinal tissue shown in FIG. 9, the histology of the intestine lengthened by the device is intact.

### EXAMPLE 2

An organ lengthening device was fabricated as a spring in a configuration similar to device 10 of FIG. 1, and made from a biodegradable polymer (polycaprolactone (PCL)). Spring characteristics were selected to create proportional increases in spring constant and size using Hooke's law. Spring diameters D measured 7 to 10 mm and uncompressed lengths L₁ measured 50 to 70 mm. Two springs were compressed and placed into size 13 gelatin capsules (Electron Microscopy Sciences, Hatsfield, Pa.) for delayed spring expansion (see capsule 60 shown in FIG. 8).

Female mini Yucatan pigs Sus scrofa, 4 to 6 weeks old, weighing at least 5 kg were intubated, anesthetized with inhaled oxygen, and vaporized isoflurane (N=12). A midline laparotomy incision was used to enter the abdomen, and the jejunum was transected 30 cm from the ligament of Trietz. A PCL spring with a spring constant between 6 and 15 N/m was placed approximately 10 cm distally into the jejunum. Once the spring was placed in enteric continuity, the transected jejunum was repaired with an end-to-end anastomosis with 4-0 Prolene suture (Ethicon, Johnson & Johnson; Somerville, N.J.) in a simple interrupted fashion to restore intestinal continuity. Full thickness 4-0 Prolene marking stitches were placed adjacent to the ends of the spring for measuring of lengthened jejunum upon specimen retrieval. The bowel was placed back into the abdomen and the abdominal wall was closed in layers.

Multiple techniques of placing the spring in-continuity were evaluated and most except for two springs were fixated to jejunal wall with full-thickness 4-0 Prolene running suture around the ends of the spring. Non-compressed, partially compressed, and compressed springs were evaluated. The following was the order of the different kinds of springs placed in-continuity:

A) Non-compressed spring: Two springs were placed in-continuity in a non-compressed state. Suture fixation at the ends of the spring and suture fixation along the entire length of the spring.

B) Partially compressed spring: Three springs were placed in-continuity in a partially compressed state with 5-0 silk suture (Ethicon, Johnson & Johnson; Somerville, N.J.) tied in the middle of the spring that was cut for immediate deployment after suture fixation.

C) Compressed spring: Seven springs were placed in-continuity in a compressed state with the use of a high-friction surface adhesive (3M, St. Paul, Minn.) wrapped around the entire length of the spring. The high-friction surface adhesive had villi-like surface features 14 that were 200 μm in height as illustrated in the micrograph image of FIG. 8. Of these springs, four had suture fixation at the ends, one did not have suture fixation, two had the adhesive sutured to the spring with 4-0 Prolene running suture and encapsulated in a size 13 gelatin capsule with suture fixation at the ends.

In a subset of animals (N=4), after 1 week post-operatively, another midline laparotomy was performed to assess for spring migration and lengthening. Intestinal segments were measured and carefully replaced back into the abdomen and abdominal wall was closed in layers.

In another subset of animals (N=8), to avoid a subsequent laparotomy, metal clips were placed on each end of the spring and on each end of the mesentery adjacent to the spring ends in order to measure spring lengthening on weekly abdominal x-rays (FIG. 5).

Lengthened jejunal segments were retrieved after 1 to 4 weeks and measured for final intestinal lengthening. Animal weights were recorded.

Lengthened and normal jejunal tissues were fixed in 10% buffered formalin overnight followed by embedding in paraffin. Tissue was aligned in perpendicular cross sections. Tissue blocks were cut into 5 μm sections and stained with hematoxylin and eosin. Sections were examined and recorded at 4× and 10× magnification using light microscopy (Olympus Corporation, Waltham, Mass.). Muscularis propria thickness and crypt depth

Data were expressed as mean values±standard deviations. Two-tailed, paired and unpaired Student's t-tests were used for statistical analyses where appropriate. All animals survived spring placement in jejunal continuity. All animals tolerated in-continuity lengthening without bowel obstruction (N=12) for up to 29 days. In-continuity jejunum with compressed springs demonstrated intestinal lengthening by 1.47-fold±0.11 (N=5). Five springs had detached prematurely and lengthening could not be accurately assessed. Animals demonstrated weight gain 0.7±0.4 kg at 2 weeks post-operatively.

On repeat laparotomy on day 14, the non-compressed spring with suture fixation at the ends of the spring had detached prematurely, whereas the spring with suture fixation along the entire length of the spring had not. These jejunal segments had not lengthened.

The partially compressed spring had not migrated up to day 23. However, fixation sutures at the end of the springs had partially eroded through the intestinal wall. Jejenual segments had lengthened to 1.47-fold±0.15.

The high-friction surface spring without suture fixation had migrated on repeat laparotomy on day 14. Four springs with the high-friction surface with suture fixation at the ends had detached prematurely but were still present endoluminally up to day 22. These springs had partial expansion immediately after suture fixation. Upon specimen retrieval, the fixation sutures at the end of these springs had also partially eroded through the intestinal wall. The high-friction adhesive had detached from the surface of the spring, and the villi-like features of the adhesive had accumulated mucous in between the structures, rendering the spring surface smooth and slippery.

The adhesive was subsequently sutured onto the spring to prevent detachment from the spring surface, and the spring was encapsulated in a gelatin capsule for delayed expansion. These two springs were present endoluminally up to 29 days, although one spring had locally migrated. The adhesive was still attached to the spring surface. Fixation sutures still partially eroded through the intestinal wall. The spring with local migration was also distorted from its original form. Of the two springs that had not migrated, the jejunal segment lengthened to 1.47-fold±0.01.

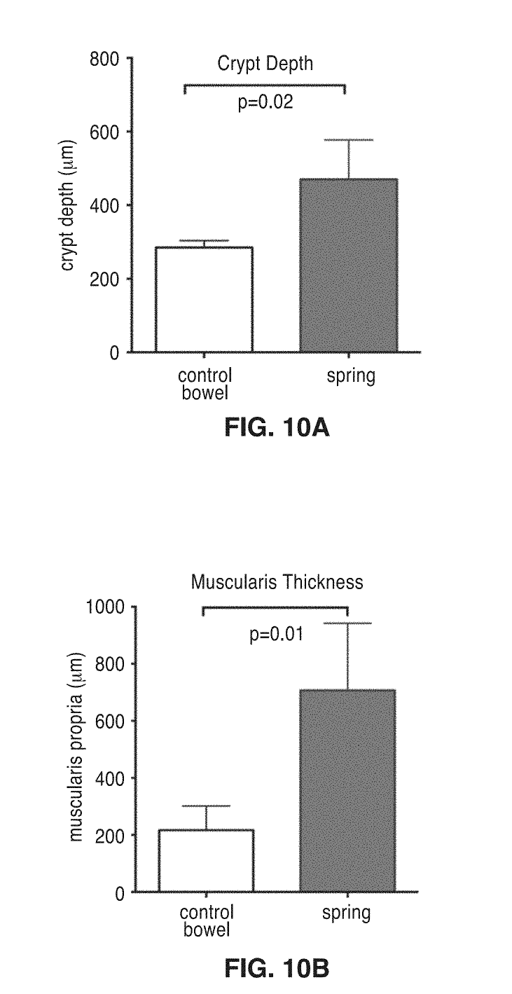

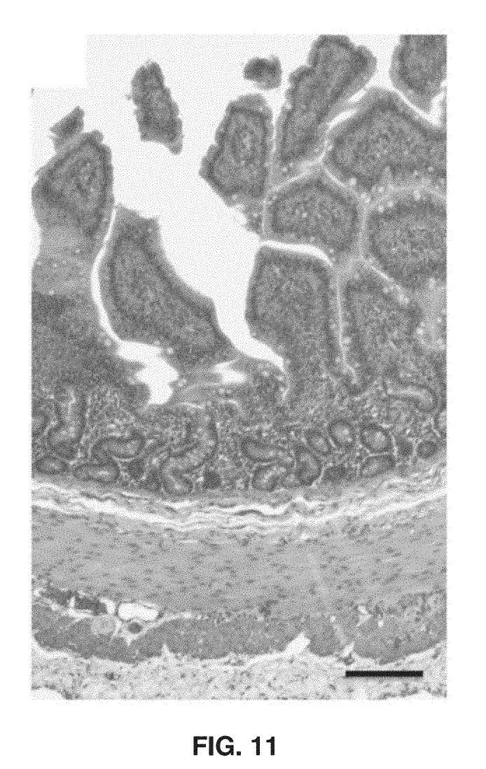

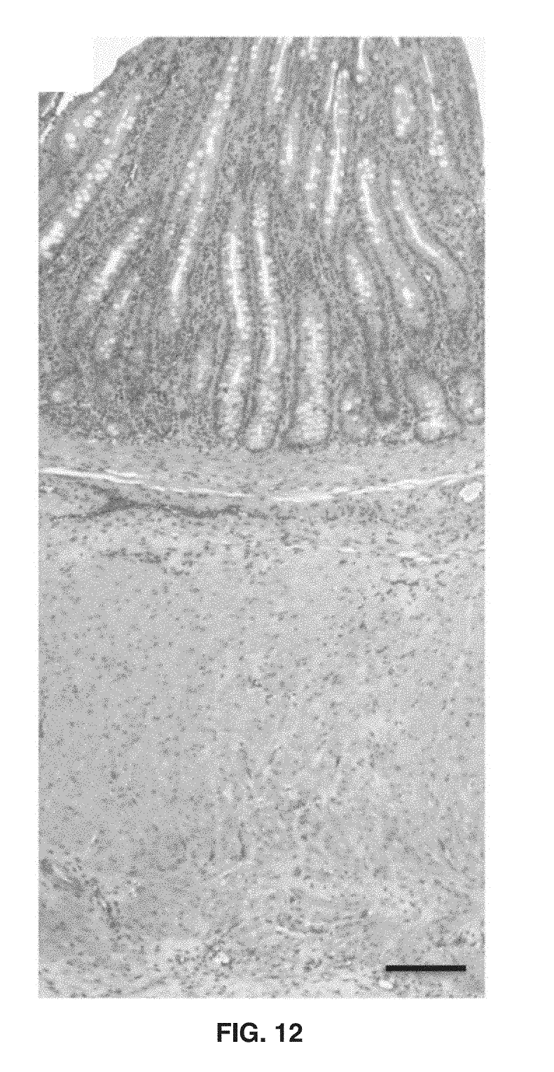

In-continuity jejunum showed significantly increased crypt depth (707±235 versus 247±96 μm, p=0.02) (as seen in FIG. 10A) and muscularis thickness (470±10 versus 285±19 μm, p=0.01) (as seen in FIG. 10B) in comparison to normal jejunum. Spring-mediated lengthened segments demonstrate significantly increased crypt depth and muscularis propria thickness relative to control jejunum. FIG. 11 shows a light microscopy image of hematoxylin and eosin-stained normal (non-lengthened) jejunum at 4× magnification. FIG. 12 shows a light microscopy image of hematoxylin and eosin-stained lengthened jejunum at 4× magnification.

The above results showed the efficacy for a completely endoluminal device for distraction enterogenesis. The device of the technology disclosed herein may be implemented via endoscopic delivery, or manual delivery into a stoma, neither of which require additional surgeries. The use of a more complex procedure to anchor the spring in this study was used purely to test the concept of evenly distributing the force of distraction along the spring. Additional spring surface modifications may be implemented to provide bowel wall coupling in a purely endoluminal fashion, thus avoiding the need for suture fixations in the future. The above results demonstrate that in-continuity spring lengthening systems and methods of the technology disclosed herein is safe and feasible.

The self-expanding endoluminal springs with and without high friction surface features placed in-continuity demonstrated intestinal lengthening without obstruction or other complications. Full-thickness suture fixation and high-friction surface adhesive facilitated lengthening without compromising luminal flow.

Although these models were effective for intestinal lengthening, the clinical impact was limited by tissue loss during restoration into continuity, significant bowel manipulation, and multiple anastomoses. These limitations may be overcome by a model that places springs directly into functional enteric continuity for intestinal lengthening.

The different anchoring techniques from this study showed that force exerted only at the ends of the spring led to suture erosion through the intestinal wall and early spring detachment. Once the spring is detached, it remained in the intestines and was removed in the fecal stream without causing any complication such as obstruction or perforation. However, when the forces were evenly distributed throughout the spring with suture fixation throughout the entire length of the spring, the spring did not migrate.

Two dimensions of intestinal growth in the length as well as the muscularis thickness were observed. The histologic results of increased muscularis thickness and crypt depth are consistent with previously reported findings. Increased muscularis thickness and crypt depth are characteristic features of tissue subjected to mechanical force as seen in previous studies. In prior studies, despite the increase in muscularis thickness during lengthening, the muscularis returned to normal levels after removal of the spring and intestinal restoration. Furthermore, intestinal motility and absorptive function remained intact.

From the description herein, it will be appreciated that that the present disclosure encompasses multiple embodiments which include, but are not limited to, the following:

1. A mechanical distension apparatus for treating a luminal organ, comprising: an elongate, tubular structure configured to be inserted into a luminal segment of the intestines, esophagus or vagina at a treatment location within the luminal segment; the tubular structure comprising a central axial channel configured to allow normal operation of said luminal organ; said tubular structure comprising a plurality of spring coils disposed between first and second ends said tubular structure such that the said tubular structure is compressible along a longitudinal axis between said first and second ends to form an axially compressed configuration; said spring coils comprising an abluminal surface with a plurality of biodegradable anchors disposed on the surface that are configured to engage an internal wall of the luminal segment at said treatment location while in said axially compressed configuration; wherein the tubular structure is biased to elongate to an expanded configuration, said bias configured to impart a force on the luminal segment at said treatment location to lengthen the luminal segment at said location; and wherein the anchors are degradable over time such that the anchors detach from the internal wall of the luminal segment after expansion of the tubular structure.

2. The apparatus of any preceding embodiment: said tubular structure being formed from a shape memory material; wherein the tubular structure is biased to elongate to the expanded configuration by memory effect.

3. The apparatus of any preceding embodiment, wherein said plurality of biodegradable anchors are disposed across the length the spring coils.

4. The apparatus of any preceding embodiment, said tubular structure having at least two spaced apart anchor portions configured to engage an internal wall of the luminal segment at said treatment location while in said axially compressed configuration.

5. The apparatus of any preceding embodiment, wherein said two spaced apart anchor portions are configured to compress into the radially compressed configuration during delivery into the luminal segment, and expand into a radially expanded configuration to engage the internal wall of the luminal segment.

6. The apparatus of any preceding embodiment, further comprising: an absorbable retaining element configured to retain the tubular structure in its axially compressed configuration; wherein the retaining element is configured to dissolve after a period of time within the lumen to free the tubular structure to impart said force on said lumen.

7. The apparatus of any preceding embodiment, wherein plurality of biodegradable anchors comprise micron-size features extending from the abluminal surface.

8. The apparatus of any preceding embodiment, wherein plurality of biodegradable anchors comprise a first set of anchors disposed in a first direction and a second set of anchors disposed in a second direction opposing the first direction.

9. The apparatus of any preceding embodiment, wherein the abluminal surface is coated with an adhesive configured to bond with at least a portion of the luminal segment.

10. The apparatus of any preceding embodiment, wherein the adhesive is mixed within a compound comprising a biodegradable polymer.

11. The apparatus of any preceding embodiment, wherein the at least two spaced apart anchor portions comprises a ratcheting member configured to conform to a radius of the luminal section.

12. A mechanical distension system for treating a luminal organ, comprising: an elongate, tubular structure configured to be inserted into a luminal segment of the intestines, esophagus or vagina at a treatment location within the luminal segment; the tubular structure comprising a central axial channel configured to allow normal operation of said luminal organ; said tubular structure comprising a plurality of spring coils disposed between first and second ends said tubular structure such that the said tubular structure is compressible along a longitudinal axis between said first and second ends to form an axially compressed configuration; said spring coils comprising an abluminal surface with a plurality of biodegradable anchors disposed on the surface that are configured to engage an internal wall of the luminal segment at said treatment location while in said axially compressed configuration; wherein the tubular structure is biased to elongate to an expanded configuration, said bias configured to impart a force on the luminal segment at said treatment location to lengthen the luminal segment at said location; wherein the anchors are degradable over time such that the anchors detach from the internal wall of the luminal segment after expansion of the tubular structure; and an absorbable retaining element configured to retain the tubular structure in its axially compressed configuration; wherein the retaining element is configured to dissolve after a period of time within the lumen to free the tubular structure to impart said force on said lumen.

13. The apparatus of any preceding embodiment, wherein said plurality of biodegradable anchors are disposed across the length the spring coils.

14. The apparatus of any preceding embodiment, wherein the plurality of biodegradable anchors comprise micron-size features extending from the abluminal surface.

15. The apparatus of any preceding embodiment, wherein the plurality of biodegradable anchors comprise a first set of anchors disposed in a first direction and a second set of anchors disposed in a second direction opposing the first direction.

16. The apparatus of any preceding embodiment, wherein the abluminal surface is coated with an adhesive configured to bond with at least a portion of the luminal segment.

17. The apparatus of any preceding embodiment, wherein the adhesive is mixed within a compound comprising a biodegradable polymer.

18. A method for mechanically distending a luminal organ, comprising: providing an elongate, tubular structure comprising a central axial channel configured to allow normal operation of said luminal organ; said tubular structure comprising a plurality of spring coils disposed between first and second ends of said tubular structure such that the said tubular structure is compressible along a longitudinal axis between said first and second ends to form an axially compressed configuration, the tubular structure being biased to elongate to an expanded configuration, the spring coils comprising an abluminal surface with a plurality of biodegradable anchors disposed on the abluminal surface; disposing the tubular structure in its axially compressed configuration with an absorbable retaining element; inserting the tubular structure in its axially compressed configuration into a luminal segment of the intestines, esophagus or vagina at a treatment location within the luminal segment; dissolving the absorbable retaining element after a period of time within the lumen to free the tubular structure; engaging an internal wall of the luminal segment at said treatment location with the biodegradable anchors while in said axially compressed configuration; imparting a force on the luminal segment at said treatment location to lengthen the luminal segment at said location; and degrading the anchors a such that the anchors detach from the internal wall of the luminal segment after expansion of the tubular structure.

19. The method of any preceding embodiment, wherein said plurality of biodegradable anchors are disposed across the length the spring coils.

20. The method of any preceding embodiment, wherein the plurality of biodegradable anchors comprise micron-size features extending from the abluminal surface.

21. The method of any preceding embodiment, wherein the plurality of biodegradable anchors comprise a first set of anchors disposed in a first direction and a second set of anchors disposed in a second direction opposing the first direction.

22. The method of any preceding embodiment, wherein the abluminal surface is coated with an adhesive configured to bond with at least a portion of the luminal segment.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A mechanical distension apparatus for treating a luminal organ, comprising:
an elongate, tubular structure configured to be inserted into a luminal segment of the intestines, esophagus or vagina at a treatment location within the luminal segment;
the tubular structure comprising a central axial channel configured to allow normal operation of said luminal organ;
said tubular structure comprising a plurality of spring coils disposed between first and second ends of said tubular structure such that the said tubular structure is compressible along a longitudinal axis between said first and second ends to form an axially compressed configuration;
said spring coils comprising an abluminal surface with a plurality of biodegradable anchors disposed on the surface that are configured to engage an internal wall of the luminal segment at said treatment location while in said axially compressed configuration;
wherein the tubular structure is biased to elongate to an expanded configuration, said bias configured to impart a force on the luminal segment at said treatment location to lengthen the luminal segment at said location; and
wherein the anchors are degradable over time such that the anchors detach from the internal wall of the luminal segment after expansion of the tubular structure.

2. An apparatus as recited in claim 1:
said tubular structure being formed from a shape memory material;
wherein the tubular structure is biased to elongate to the expanded configuration by memory effect.

3. An apparatus as recited in claim 1, wherein said plurality of biodegradable anchors are disposed across the length the spring coils.

4. An apparatus as recited in claim 1, said tubular structure having at least two spaced apart anchor portions configured to engage an internal wall of the luminal segment at said treatment location while in said axially compressed configuration.

5. An apparatus as recited in claim 4, wherein said two spaced apart anchor portions are configured to compress into the radially compressed configuration during delivery into the luminal segment, and expand into a radially expanded configuration to engage the internal wall of the luminal segment.

6. An apparatus as recited in claim 1, further comprising:
an absorbable retaining element configured to retain the tubular structure in its axially compressed configuration;
wherein the retaining element is configured to dissolve after a period of time within the lumen to free the tubular structure to impart said force on said lumen.

7. An apparatus as recited in claim 1, wherein the plurality of biodegradable anchors comprise micron-size features extending from the abluminal surface.

8. An apparatus as recited in claim 1, wherein the plurality of biodegradable anchors comprise a first set of anchors disposed in a first direction and a second set of anchors disposed in a second direction opposing the first direction.

9. An apparatus as recited in claim 1, wherein the abluminal surface is coated with an adhesive configured to bond with at least a portion of the luminal segment.

10. An apparatus as recited in claim 9, wherein the adhesive is mixed within a compound comprising a biodegradable polymer.

11. An apparatus as recited in claim 4, wherein the at least two spaced apart anchor portions comprises a ratcheting member configured to conform to a radius of the luminal segment.

12. A mechanical distension system for treating a luminal organ, comprising:
an elongate, tubular structure configured to be inserted into a luminal segment of the intestines, esophagus or vagina at a treatment location within the luminal segment;
the tubular structure comprising a central axial channel configured to allow normal operation of said luminal organ;
said tubular structure comprising a plurality of spring coils disposed between first and second ends said tubular structure such that the said tubular structure is compressible along a longitudinal axis between said first and second ends to form an axially compressed configuration;
said spring coils comprising an abluminal surface with a plurality of biodegradable anchors disposed on the surface that are configured to engage an internal wall of the luminal segment at said treatment location while in said axially compressed configuration;
wherein the tubular structure is biased to elongate to an expanded configuration, said bias configured to impart a force on the luminal segment at said treatment location to lengthen the luminal segment at said location;
wherein the anchors are degradable over time such that the anchors detach from the internal wall of the luminal segment after expansion of the tubular structure; and
an absorbable retaining element configured to retain the tubular structure in its axially compressed configuration;
wherein the retaining element is configured to dissolve after a period of time within the lumen to free the tubular structure to impart said force on said lumen.

13. An apparatus as recited in claim 12, wherein said plurality of biodegradable anchors are disposed across the length the spring coils.

14. An apparatus as recited in claim 12, wherein the plurality of biodegradable anchors comprise micron-size features extending from the abluminal surface.

15. An apparatus as recited in claim 12, wherein the plurality of biodegradable anchors comprise a first set of anchors disposed in a first direction and a second set of anchors disposed in a second direction opposing the first direction.

16. An apparatus as recited in claim 12, wherein the abluminal surface is coated with an adhesive configured to bond with at least a portion of the luminal segment.

17. An apparatus as recited in claim 16, wherein the adhesive is mixed within a compound comprising a biodegradable polymer.

18. A method for mechanically distending a luminal organ, comprising:
providing an elongate, tubular structure comprising a central axial channel configured to allow normal operation of said luminal organ, said tubular structure comprising a plurality of spring coils disposed between first and second ends of said tubular structure such that the said tubular structure is compressible along a longitudinal axis between said first and second ends to form an axially compressed configuration, the tubular structure being biased to elongate to an expanded configuration along a longitudinal axis, the spring coils comprising an abluminal surface with a plurality of biodegradable anchors disposed on the abluminal surface;
disposing the tubular structure in its axially compressed configuration with an absorbable retaining element;
inserting the tubular structure in its axially compressed configuration into a luminal segment of the intestines, esophagus or vagina at a treatment location within the luminal segment;
dissolving the absorbable retaining element after a period of time within the lumen to free the tubular structure;
engaging an internal wall of the luminal segment at said treatment location with the plurality of biodegradable anchors while in said axially compressed configuration;
imparting a force on the luminal segment at said treatment location to lengthen the luminal segment at said location; and
degrading the anchors a such that the anchors detach from the internal wall of the luminal segment after expansion of the tubular structure.

19. A method as recited in claim 18, wherein said plurality of biodegradable anchors are disposed across the length the spring coils.

20. A method as recited in claim 18, wherein the plurality of biodegradable anchors comprise micron-size features extending from the abluminal surface.

21. A method as recited in claim 18, wherein the plurality of biodegradable anchors comprise a first set of anchors disposed in a first direction and a second set of anchors disposed in a second direction opposing the first direction.

22. A method as recited in claim 18, wherein the abluminal surface is coated with an adhesive configured to bond with at least a portion of the luminal segment.
